(19) United States
(12) Patent Application Publication
(10) Pub. No.: US 2015/0374244 A1
(43) Pub. Date: Dec. 31, 2015

(54) APPARATUS FOR CONTINUOUSLY AND AUTOMATICALLY MEASURING PULSE WAVE AND METHOD FOR MEASURING BLOOD PRESSURE

(71) Applicant: K-HEALTHWEAR CO., LTD., Seo-gu Daejeon (KR)

(72) Inventors: Hoi Jun YOO, Daejeon (KR); Tae-Hwan ROH, Daejeon (KR); Kwon Joon LEE, Daejeon (KR)

(73) Assignee: K-HEALTHWEAR CO., Ltd., Daejeon (KR)

(21) Appl. No.: 14/765,346

(22) PCT Filed: Feb. 5, 2014
(86) PCT No.: PCT/KR2014/000964

§ 371 Date: Aug. 3, 2015

(30) Foreign Application Priority Data
Feb. 8, 2013 (KR) 10-2013-0014455

(51) Int. Cl. A61B 5/0245; A61B 7/04; A61B 5/021; A61B 5/00; A61B 5/0402; A61B 5/053

(52) U.S. Cl. CPC A61B 5/0245; A61B 5/0402; A61B 5/053; A61B 5/02125; A61B 5/7235; A61B 5/6824; A61B 5/6823; A61B 7/04

(57) ABSTRACT

Disclosed is an apparatus for continuously and automatically measuring a pulse wave by a non-invasive method to know the state of a cardiovascular system. The apparatus includes an integrated measurement module, a communication power module, and a bio-measurement pad. The integrated measurement module includes an electrocardiogram measurement portion for measuring the electrocardiogram of a subject, a bioelectrical impedance measurement portion for measuring the bioelectrical impedance of the subject by a potential difference, a heart sound measurement portion for measuring the heart sound of the subject, and a controller for measuring and controlling the state of the cardiovascular system of the subject on the basis of a pulse transit time calculated by the electrocardiogram signal measured at the electrocardiogram measurement portion, the bioelectrical impedance signal measured at the bioelectrical impedance measurement portion, and the heart sound signal measured at the heart sound measurement portion.

### TECHNICAL FIELD

The present invention relates to an apparatus for measuring a pulse wave continuously and automatically, which measures a pulse wave using a non-invasive method so that the state of a cardiovascular system can be checked, and a method for measuring blood pressure.

### BACKGROUND ART

In general, a method for measuring blood pressure includes a vascular sound stethoscopy using a tourniquet and a stethoscope.

The vascular sound stethoscopy is a method of winding the tourniquet on the brachial (the upper part of an arm), pressing air pressure, and auscultating a vascular sound through the stethoscope. An ordinal person has a difficult in measuring blood pressure using this method because blood pressure needs to be measured through a vascular sound and the method is performed by educated medical personnel rather than an ordinary person.

Recently, there has been disclosed a blood pressure measurement device for measuring blood pressure using an oscillometric method so that blood pressure can be easily measured at home.

In the blood pressure measurement device using the oscillometric method, an ordinary person could easily measure blood pressure because a machine measures blood pressure by automatically checking a vascular sound. However, a testee feels inconvenient because pressure is applied to an arm using a tourniquet as in the vascular sound stethoscopy, and the continuous measurement of blood pressure was impossible because rest needs to be taken for a specific time for re-measurement.

In order to solve the problems, in a prior art, there has been disclosed “Apparatus for measuring pulse wave velocity and method thereof, and diagnosis system including the same”, which are capable of measuring blood pressure in a continuous and non-invasive manner as in Korean Patent No. 10-1056016.

The conventional apparatus for measuring pulse wave velocity is an apparatus for measuring the pulse wave velocity of a testee, including a bioimpedance signal measurement unit for measuring a bioimpedance signal generated based on a test current transferred to part of the body of the testee; an electrocardiogram signal measurement unit for measuring the electrocardiogram signal of the testee; and a data processing unit for measuring the pulse wave velocity of the testee based on the bioimpedance signal and the electrocardiogram signal. The bioimpedance signal measurement unit includes a test current generation unit for generating a test current transferred to part of the body of the testee; a bioimpedance signal electrode unit for transferring the test current to the part of the body of the testee and detecting a potential difference of the part of the body of the testee generated based on the transferred test current; a bioimpedance signal amplification unit for generating an amplification bioimpedance signal based on the detected potential difference; and a bioimpedance signal processing unit for demodulating and filtering the amplification bioimpedance signal and providing the bioimpedance signal.

The conventional apparatus for measuring pulse wave velocity could calculate a pulse wave transfer time using an electrocardiogram signal and a bioimpedance signal and measure the blood pressure of a testee in such a way as to derive blood pressure according to a regression equation using the pulse wave transfer time.

However, an electrocardiogram signal is only an electrical signal and is delayed for a significant time (hereinafter called as a “Pre-Ejection Period (PEP)”) until the heart is actually contracted. An error is caused if the pulse wave transfer time is measured based on the electrocardiogram signal because a pulse wave is a mechanical signal affecting the wall of a blood vessel.

Accordingly, there was a problem in that to derive blood pressure based on the pulse wave transfer time of the conventional apparatus for measuring pulse wave velocity, which uses the pulse wave transfer time, that is, a time interval between the extreme values of a bioimpedance signal, using the peak point R of the electrocardiogram signal as a reference time was inaccurate.

Furthermore, the PEP necessary to calculate a stroke volume, that is, the amount of spouted blood of the heart for one heartbeat, could not be measured.

In particular, in order to accurately measure the PEP, expensive and large-sized equipment was required. There was a problem in that a testee must move to the place where the equipment was placed because the state of a cardiovascular system could be checked only at the place where the equipment was installed.

### DISCLOSURE

### Technical Problem

The present invention has been made to solve the problems, and an object of the present invention is to provide an apparatus for measuring a pulse wave continuously and automatically, which can measure the state of the cardiovascular system of a testee easily and accurately, can be fabricated at relatively low cost, can be easily carried due to a small size, and can easily check the state of the cardiovascular system through an external terminal carried by a testee, and a method for measuring blood pressure.

### Technical Solution

An apparatus for measuring a pulse wave continuously and automatically in accordance with an embodiment of the present invention for achieving the object includes an integrated measurement module including an electrocardiogram measurement unit which measures electrocardiogram of a testee, a bioelectrical impedance measurement unit which measures bioelectrical impedance of the testee based on a potential difference, a cardiac sound measurement unit which measures a cardiac sound of the testee, and a controller which measures the state of a cardiovascular system of the testee based on a pulse transit time (PTT′) calculated based on the electrocardiogram signal measured by the electrocardiogram measurement unit, the bioelectrical impedance signal measured by the bioelectrical impedance measurement unit, and the cardiac sound signal measured by the cardiac sound measurement unit; a first communication power module including a first wireless communication unit which is electrically connected to the integrated measurement module and wirelessly sends and receives information of the integrated measurement module and information of an external terminal and a first power supply unit which supplies power to the first wireless communication unit and the measurement module; and a biomeasurement pad in which the integrated measurement module and the first communication power module are seated and which includes bioelectrodes electrically connected to the bioelectrical impedance measurement unit.

The controller may calculate the pulse transit time (PTT′) using the pulse transit time (PTT′)=PTT−PEP. (In this case, a PTT is a time interval between a peak point R in the electrocardiogram signal and a highest point or lowest point in the bioelectrical impedance signal, and the PEP is a time interval between the peak point R in the electrocardiogram signal and a first highest point S1 of the cardiac sound signal).

The apparatus may further include a cuff which fixes the integrated measurement module and the first communication power module in such a way as to surround part of the body of the testee.

The biomeasurement pad may be attached to a wrist portion of the testee.

The first communication power module may be detachably coupled to the integrated measurement module and the cuff and may be reused.

The first power supply unit may include a warning unit which warns the state of power supply.

The apparatus may further include an electrocardiogram pad including an electrocardiogram electrode which is electrically connected to the electrocardiogram measurement unit and detects the electrocardiogram signal for the testee based on the potential difference and a cardiac sound sensor which is electrically connected to the cardiac sound measurement unit and detects the cardiac sound signal for the testee. The electrocardiogram pad may be attached to a portion in which the heart of the testee is placed.

The electrocardiogram pad may include a second communication power module including a second wireless communication unit which wirelessly sends the electrocardiogram signal and the cardiac sound signal and a second power supply unit which supplies power to the second wireless communication unit, the electrocardiogram electrodes, and the cardiac sound sensor.

The second communication power module may be detachably coupled to the electrocardiogram pad and may be reused.

The second wireless communication unit may communicate with the first wireless communication unit over a Personal Area Network (PAN) or a Body Area Network (BAN).

The controller may receive information about the body of the testee from the external terminal and measure the state of the cardiovascular system of the testee based on the information about the body of the testee.

The external terminal may include a user terminal used by the testee and a tester terminal which directly receives information about the state of the cardiovascular system of the testee through the first wireless communication unit or receives the information through the user terminal, receives feedback information from a tester, and sends the information to the user terminal.

A method for measuring blood pressure in accordance with an embodiment of the present invention includes steps of measuring the electrocardiogram signal of a testee; measuring the bioelectrical impedance signal of the testee based on a potential difference; measuring the cardiac sound signal of the testee; and measuring the state of a cardiovascular system of the testee based on a pulse transit time calculated based on the electrocardiogram signal, the bioelectrical impedance signal, and the cardiac sound signal. The step of measuring the state of the cardiovascular system of the testee includes calculating a pulse transit time (PTT′) using the pulse transit time (PTT′)=PTT−PEP. In this case, the PTT is a time interval between a peak point R in the electrocardiogram signal and a highest point or lowest point in the bioelectrical impedance signal, and the PEP is a time interval between the peak point R in the electrocardiogram signal and a first highest point S1 of the cardiac sound signal.

The bioelectrical impedance may be measured in a wrist portion of the testee, and the electrocardiogram signal may be measured in a heart portion of the testee.

### Advantageous Effects

In accordance with the present invention, the apparatus for measuring a pulse wave continuously and automatically can measure accurate blood pressure because it derives blood pressure by calculating a pulse wave transfer time using a cardiac sound signal, an electrocardiogram signal, and a bioelectrical impedance signal and can be easily installed in the body of a testee due to a relatively simple configuration.

Furthermore, the apparatus for measuring a pulse wave continuously and automatically can check the state of a cardiovascular system regardless of a place because it has a low production cost due to a relatively simple configuration and can be easily carried.

Furthermore, a testee can easily check the measured state of a cardiovascular system through an external terminal through wireless communication and can easily receive feedback information from medical personnel.

Furthermore, damage attributable to the wrinkling of power lines can be prevented because power lines can be omitted through the wireless exchange of information between the electrocardiogram pad and the controller.

Furthermore, there is an advantage in that repetitive reuse is possible because the first wireless communication unit is detachably coupled to the integrated measurement module.

Furthermore, one stroke volume of the heart can be measured accurately and easily because a PEP is measured using a cardiac sound signal and an electrocardiogram signal.

### DESCRIPTION OF DRAWINGS

FIG. 1 is a diagram illustrating the state in which an apparatus for measuring a pulse wave continuously and automatically in accordance with an embodiment of the present invention has been attached to a testee.

FIG. 2 is a perspective view schematically illustrating the apparatus for measuring a pulse wave continuously and automatically in accordance with an embodiment of the present invention.

FIG. 3 is a perspective view schematically illustrating the apparatus for measuring a pulse wave continuously and automatically in accordance with an embodiment of the present invention and is a diagram illustrating the state in which a cuff has been connected to the apparatus.

FIG. 4 is a diagram schematically illustrating the configuration of the apparatus for measuring a pulse wave continuously and automatically in accordance with an embodiment of the present invention.

FIG. 5 is a diagram schematically illustrating the configuration of the apparatus for measuring a pulse wave continuously and automatically in accordance with an embodiment of the present invention and illustrates the state in which a second communication power module is combined with an electrocardiogram pad.

FIG. 6 is a diagram illustrating a communication state between the apparatus for measuring a pulse wave continuously and automatically and an external terminal in accordance with an embodiment of the present invention.

FIG. 7 is a diagram illustrating measured signals for describing a method for measuring blood pressure in accordance with an embodiment of the present invention.

[Description of reference numerals]

100: apparatus for measuring a pulse wave continuously

| | |
|---|---|
| and automatically | 110: integrated measurement module |

| 111: electrocardiogram measurement unit | |
|---|---|
| 111a: electrocardiogram signal amplification unit | |
| 111b: electrocardiogram signal filtering unit | |
| 111c: electrocardiogram signal conversion unit | |
| 113: cardiac sound measurement unit | |
| 113a: cardiac sound signal amplification unit | |
| 113b: cardiac sound signal filtering unit | |
| 113c: cardiac sound signal conversion unit | |
| 115: bioelectrical impedance measurement unit | |
| 115a: bio-signal amplification unit | |
| 15b: bio-signal filtering unit | |
| 115c: bio-signal conversion unit | |
| 117: controller | 120: first communication power module |

| 121: first wireless commmunication unit | |
|---|---|
| 123: first power supply unit | |
| 125: warning unit | 130: biomeasurement pad |
| 131: bioelectrode | 140: electrocardiogram pad |

| 141: electrocardiogram electrode | |
|---|---|
| 143: cardiac sound sensor | |
| 150: cuff | 151: fastening means |

| 160: second communication power module | |
|---|---|
| 161: second wireless communication unit | |
| 163: second power supply unit | 170: external terminal |

| | |
|---|---|
| 171: testee terminal | 173: feedback terminal |

### MODE FOR INVENTION

Hereinafter, embodiments of the present invention are described with reference to the accompanying drawings.

First, an apparatus 100 for measuring a pulse wave continuously and automatically in accordance with an embodiment of the present invention is an apparatus capable of measuring the state of the cardiovascular system of a testee, for example, blood pressure and the degree of hardening of the arteries in a non-invasive manner.

In this specification, coupling means is means that is detachably coupled and may be implemented using a known coupling structure in which a male is inserted into a female and the vice versa, such as a protrusion and a groove, for example, a known coupling member in which a male is inserted into a female and the vice versa, such as a Velcro tape or a snap button, for example, or a known adhesive member, such as adhesives.

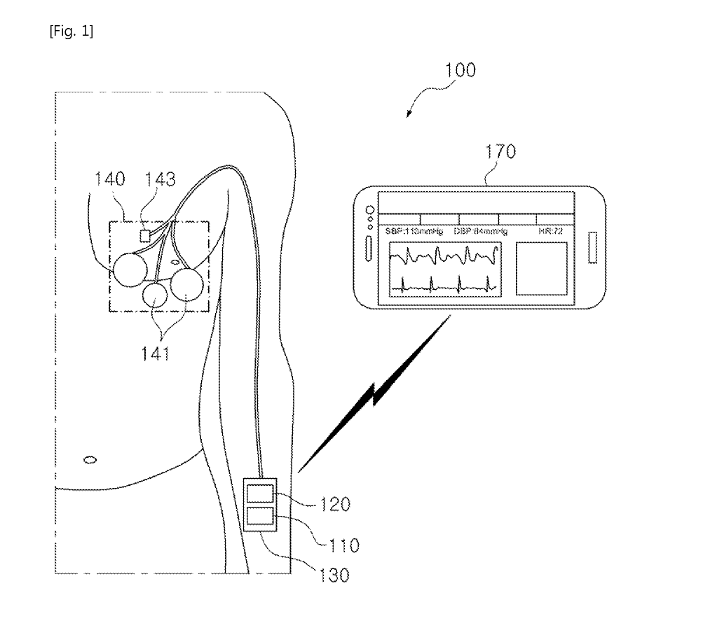

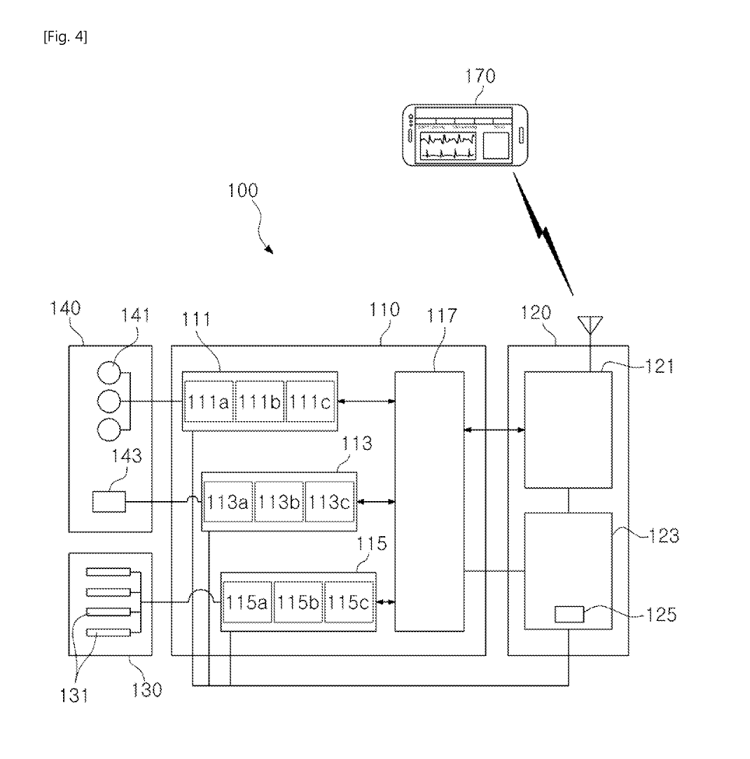

As illustrated in FIGS. 1 and 4, the apparatus 100 for measuring a pulse wave continuously and automatically in accordance with an embodiment of the present invention may include an electrocardiogram measurement unit 111.

The electrocardiogram measurement unit 111 measures the electrocardiogram of a testee and may measure an electrocardiogram signal ECG displayed in the form of a continuous pulse wave based on a potential difference.

In this case, the electrocardiogram is obtained by deriving an active current generated in a myocardium depending on the pulsation of the heart to the two places of the body of a testee and recording the active currents using an amperemeter, and means the record of the active current of the myocardium.

Furthermore, the electrocardiogram measurement unit 111 may measure an electrocardiogram signal at a portion where the heart is placed using electrocardiogram electrodes 141 attached to a portion where the heart of a testee is placed.

Meanwhile, the electrocardiogram signal measured by the electrocardiogram measurement unit 111 may be divided into a P wave, a QRS group, and a T wave. The first part of the P wave denotes the depolarization of a right atrium, the rear part of the P wave denotes the depolarization of a left atrium. Normally, the P wave is generated during a ventricular diastolic period.

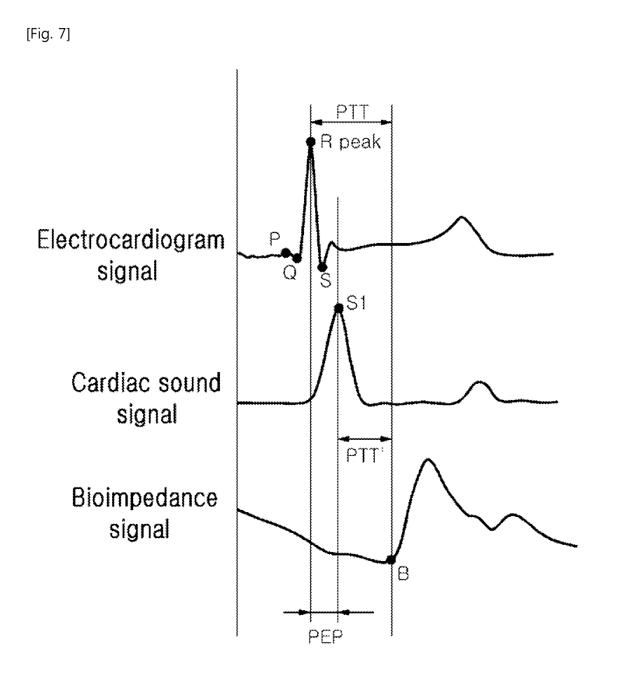

Furthermore, the QRS group is a waveform complexly formed of a group of a Q wave, an R wave, and an S wave. A first downstream wave connected to the P wave is called the Q wave. A first upstream wave is called the R wave. A downstream wave connected to the R wave is called the S wave. QRS is generated within a short time (normally 0.06 second-0.10 second). The QRS wave denotes the depolarization of left/right ventricles (refer to FIG. 7).

Furthermore, the T wave denotes the normal repolarization of a ventricle.

Such an electrocardiogram signal may be used to check the state of the heart in a form for analyzing the regularity of appearance frequency of each wave, a waveform, and the height of a wave. The accuracy of blood pressure can be improved by obtaining a heartbeat using the occurrence time of each wave and approximating one stroke volume from the heartbeat.

Meanwhile, the electrocardiogram measurement unit 111 may include an electrocardiogram signal amplification unit 111a, an electrocardiogram signal filtering unit 111b, and an electrocardiogram signal conversion unit 111c. The electrocardiogram measurement unit 111 may be implemented using an electronic circuit.

The electrocardiogram signal amplification unit 111a may amplify an electrocardiogram signal measured from a testee. The electrocardiogram signal filtering unit 111b may obtain a filtered electrocardiogram signal by removing a noise included in the electrocardiogram signal amplified by the electrocardiogram signal amplification unit 111a or extracting an electrocardiogram signal of a specific band.

Furthermore, the electrocardiogram signal conversion unit 111c may convert the analog electrocardiogram signal, obtained by the electrocardiogram signal filtering unit 111b, into a digital signal.

As illustrated in FIG. 4, the apparatus 100 for measuring a pulse wave continuously and automatically in accordance with an embodiment of the present invention may include a bioelectrical impedance measurement unit 115.

The bioelectrical impedance measurement unit 115 may measure the bioelectrical impedance signal of a testee based on a potential difference.

In this case, the bioelectrical impedance signal measures a potential difference varying depending on a given AC current and primarily measures the volume heartbeat wave of a blood vessel. The volume heartbeat wave may measure a pulse wave, that is, a pressure heartbeat wave, through the volume heartbeat wave because it has a 1:1 correlation with the pressure heartbeat of the blood vessel.

Accordingly, the state of a cardiovascular system, such as the volume of the main artery, the amount of blood, the blood distribution, the activities of the endocrine system, and the activities of the autonomic nervous system of a testee through the bioelectrical impedance signal.

Furthermore, the bioelectrical impedance measurement unit 115 may preferably obtain an impedance signal at a wrist portion through bioelectrodes 131 installed in the wrist portion to be described later.

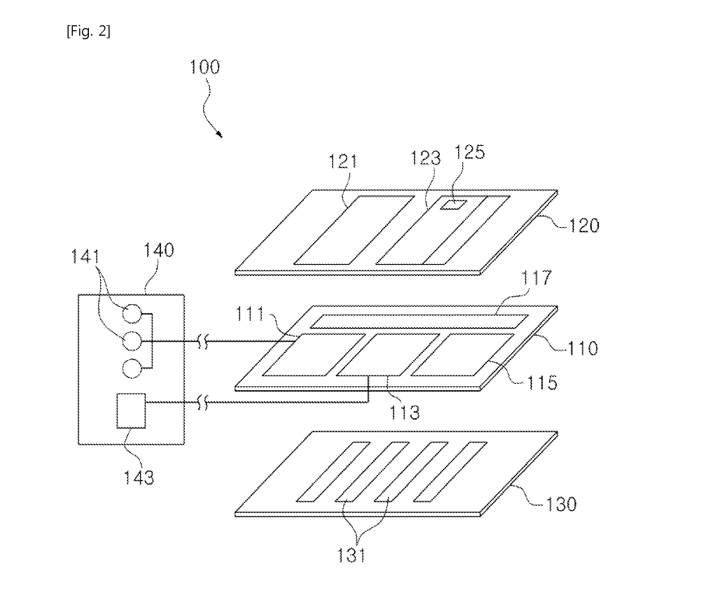

Meanwhile, the bioelectrical impedance measurement unit 115 may be configured to measure bioelectrical impedance in such a manner that four bioelectrodes 131 are attached to a wrist portion, two bioelectrodes 131 that belong to the four bioelectrodes 131 and that are placed at both edges of the wrist portion have a frequency of about 100 KHz, and a voltage induced to the remaining two bioelectrodes 131 after flowing an AC current having amplitude of several ampere (mA) to the wrist is measured (refer to FIGS. 2 to 4).

Furthermore, the bioelectrical impedance measurement unit 115 may include a bio-signal amplification unit 115a, a bio-signal filtering unit 115b, and a bio-signal conversion unit 115c. The bioelectrical impedance measurement unit 115 may be implemented using an electronic circuit.

The bio-signal amplification unit 115a may amplify a bioelectrical impedance signal obtained from a testee. The bio-signal filtering unit 115b may obtain a filtered bioelectrical impedance signal by removing a noise included in the bioelectrical impedance signal amplified by the bio-signal amplification unit 115a or extracting an impedance signal of a specific band.

Furthermore, the bio-signal conversion unit 115c may convert the analog bioelectrical impedance signal, filtered by the bio-signal filtering unit 115b, into a digital signal.

As illustrated in FIG. 4, the apparatus 100 for measuring a pulse wave continuously and automatically in accordance with an embodiment of the present invention may include a cardiac sound measurement unit 113.

The cardiac sound measurement unit may measure a cardiac sound signal that displays the contraction and expansion sound of the heart in the form of a continuous pulse wave.

In this case, the cardiac sound signal is indicative of the exercise sound of the heart. The state of a cardiovascular system, such as a heartbeat and the abnormality of the heart, can be checked through the cardiac sound signal.

Meanwhile, the cardiac sound measurement unit 113 may measure the exercise sound of the heart through a cardiac sound sensor 143 attached to a portion where the heart of a testee is placed. The cardiac sound sensor 143 may be implemented using a known microphone or piezo substance.

Furthermore, the cardiac sound measurement unit 113 may include a cardiac sound signal amplification unit 113a, a cardiac sound signal filtering unit 113b, and a cardiac sound signal conversion unit 113c. The cardiac sound measurement unit 113 may be implemented using an electronic circuit.

The cardiac sound signal amplification unit 113a amplifies a cardiac sound signal obtained from a testee. The cardiac sound signal filtering unit 113b may obtain a filtered cardiac sound signal by removing a noise included in the amplified cardiac sound signal or extracting a cardiac sound signal of a specific band.

Furthermore, the cardiac sound signal conversion unit 113c may convert the analog cardiac sound signal, filtered by the cardiac sound signal filtering unit 113b, into a digital signal.

As illustrated in FIG. 4, the apparatus 100 for measuring a pulse wave continuously and automatically in accordance with an embodiment of the present invention may include a controller 117.

The controller 117 may control the electrocardiogram measurement unit 111, the bioelectrical impedance measurement unit 115, and the cardiac sound measurement unit 113 or may check the state of the cardiovascular system of a testee by analyzing an electrocardiogram signal, a bioelectrical impedance signal, and a cardiac sound signal measured by the respective measurement units 111, 113, and 115.

Meanwhile, the controller 117 may be a controller 117 having a microprocessor form. The controller 117 may check the state of the cardiovascular system of a testee by calculating or performing a comparison on an electrocardiogram signal, a bioelectrical impedance signal, and a cardiac sound signal measured from the testee based on previously stored normal state information about the cardiovascular system (DB) and information about the body of the testee received from an external terminal.

For example, the controller 117 may measure the blood pressure of a testee using the following method for measuring blood pressure based on an electrocardiogram signal, a bioelectrical impedance signal, and a cardiac sound signal measured from the testee.

The method for measuring blood pressure in accordance with an embodiment of the present invention may be derived according to Equation 1 below.

PTT′=PTT−PEP [Equation 1]

Equation 1 is an equation for calculating a pulse transit time (PTT′) in order to derive blood pressure.

As illustrated in FIG. 7, assuming that in a repeated electrocardiogram signal, bioelectrical impedance signal, and cardiac sound signal, an interval between a peak point R (R peak), that is, the highest point of the electrocardiogram signal, and the lowest point B of the bioelectrical impedance signal is a Pulse Transit Time (PTT) and an interval between the peak point R (R peak) of the electrocardiogram signal and the highest point S1 of the cardiac sound signal is a Pre-Ejection Period (PEP), blood pressure may be derived through a regression equation based on the value of a pulse transit time (PTT′) calculated by setting a value, obtained by subtracting the PEP from the PTT, as the pulse transit time (PTT′).

In this case, in a prior art, an interval between the peak point R (R peak) of the electrocardiogram signal and the lowest point B of the bioelectrical impedance signal was simply calculated as the PTT, and blood pressure was derived through a regression equation based on the value (PTT) (a method for deriving blood pressure based on the regression equation is a known art, and a detailed description thereof is omitted).

However, a significant time is delayed until the heart is actually contracted because the peak point R (R peak) of the electrocardiogram signal is an electrical signal. Accordingly, accurate blood pressure could not be measured if blood pressure was derived simply using only the PTT as in the prior art due to the delayed time.

Accordingly, in the present invention, accurate blood pressure can be derived based on the time when the heart actually operates by calculating the PEP by measuring the time when the heart is actually contracted based on a cardiac sound time and deriving blood pressure based on a value obtained by subtracting the measured time from the PTT.

Furthermore, in the present invention, the controller 117 may derive more accurate blood pressure according to information about the body of a testee using Equation 2 below.

BP=f(PTT′)+f(cardiac sound signal)+f(electrocardiogram signal)+f(body information) [Equation 2]

In this case, BP is blood pressure, PTT′ is a pulse transit time calculated by Equation 1 and may be a heart rate per minute according to a cardiac sound signal. The body information is information about the body of a testee, and may include height, weight, a degree of obesity, and an age.

Furthermore, “+” does not mean the addition of values in terms of the number, but means that blood pressure is derived using a value derived by each function as a variable.

If blood pressure is derived according to a regression equation using Equation 2 as described above, blood pressure according to information about the body, the cardiac sound signal, and the electrocardiogram signal of a testee can be derived more accurately.

Meanwhile, the controller 117 may check the state of the cardiovascular system of a testee in a signal form in response to an electrocardiogram signal, a bioelectrical impedance signal, and a cardiac sound signal. The controller 117 may compress or encrypt a signal indicative of the state of the cardiovascular system of a testee.

Furthermore, the electrocardiogram measurement unit 111, the cardiac sound measurement unit 113, the bioelectrical impedance measurement unit 115, and the controller 117 may be configured into an integrated measurement module 110, that is, a module form formed of a single electronic circuit, for example.

As illustrated in FIG. 4, the apparatus 100 for measuring a pulse wave continuously and automatically in accordance with an embodiment of the present invention may include a first wireless communication unit 121.

The first wireless communication unit 121 may wirelessly send a signal indicative of the state of the cardiovascular system of a testee, checked by the controller 117, to an external terminal 170 to be described later or may wirelessly receive the signal of information about the body of the testee received from the external terminal 170 and send the signal to the controller 117.

In this case, the first wireless communication unit 121 may communicate with the external terminal 170 using a wireless communication method, such as Wi-Fi, Bluetooth, Zigbee, NFC, WirelessHART, a Body Area Network (BAN), a Wireless BAN (WBAN), or a Personal Area Network (PAN), such as an ultra wideband (UWB).

As illustrated in FIG. 1, the apparatus 100 for measuring a pulse wave continuously and automatically in accordance with an embodiment of the present invention may include a first power supply unit 123.

The first power supply unit 123 is electrically connected the first wireless communication unit 121 and the integrated measurement module 110, and may supply power.

Meanwhile, a portable battery may be embedded in the first power supply unit 123. The battery may be detachably coupled to the first power supply unit 123. In this case, the battery may be a disposable primary cell or a rechargeable secondary cell.

Furthermore, the first power supply unit 123 may include a warning unit 125 for warning the abnormal state of the battery. The warning unit 125 may be implemented using an LED lamp for visually giving warning or a speaker for acoustically giving warning.

Furthermore, the first wireless communication unit 121 and the first power supply unit 123 may configured into a first communication power module 120, that is, a module form formed of a single electronic circuit, for example.

In this case, if the first wireless communication unit 121 and the first power supply unit 123 are configured into the first communication power module 120, the first power supply unit 123 may be detachably coupled to the first communication power module 120 so that the first power supply unit 123 of the first communication power module 120 may be replaced or separately recharged.

In this case, the first communication power module 120 and the first power supply unit 123 may be equipped with connection terminals that may be electrically coupled or coupling means that may be detachably coupled.

As illustrated in FIGS. 1 and 4, the apparatus 100 for measuring a pulse wave continuously and automatically in accordance with an embodiment of the present invention may include a biomeasurement pad 130.

The biomeasurement pad 130 comes in contact with the body of a testee and extracts bioelectrical impedance from the body, and may be configured using fabrics of a pad form. The biomeasurement pad 130 may include the bioelectrodes 131 electrically connected to the bioelectrical impedance measurement unit 115.

Meanwhile, the bioelectrodes 131 comes in contact with the body of a testee, and may transfer an AC current generated by the bioelectrical impedance measurement unit 115 to the body of the testee and receives an electric current induced from the AC current transferred to the body.

In an embodiment, four bioelectrodes 131 have been configured. The four bioelectrodes 131 have been configured so that two bioelectrodes 131 that belong to the four bioelectrodes 131 and that are placed on both sides transfer an AC current, an electric current induced from the two bioelectrodes 131 at the center is received, and a varying voltage between an electric current output by and an electric current inputted to the bioelectrical impedance measurement unit 115 is measured.

Furthermore, as illustrated in FIG. 2, the biomeasurement pad 130 may be equipped with an adhesive layer (not illustrated) so that the biomeasurement pad 130 is easily attached to or detached from the body of a testee. The integrated measurement module 110 and the first communication power module 120 may be detachably coupled to the biomeasurement pad 130 in an overlap manner.

In this case, a connection terminal may be provided so that the integrated measurement module 110 seated on a top surface of the biomeasurement pad 130 is electrically connected to the biomeasurement pad 130, and coupling means may be provided so that the integrated measurement module 110 and the biomeasurement pad 130 are detachably coupled.

As illustrated in FIGS. 1 and 4, the apparatus 100 for measuring a pulse wave continuously and automatically in accordance with an embodiment of the present invention may include an electrocardiogram pad 140.

The electrocardiogram pad 140 comes in contact with the body of a testee and extracts electrocardiogram and a cardiac sound. The electrocardiogram pad 140 may include an electrocardiogram electrode 141 for extracting an electrocardiogram signal based on a potential difference and a cardiac sound sensor 143 for extracting a cardiac sound signal.

The electrocardiogram electrode 141 is electrically connected to the electrocardiogram measurement unit 111, and may transfer a measured electrocardiogram signal to the electrocardiogram measurement unit 111. The cardiac sound sensor 143 is electrically connected to the cardiac sound measurement unit 113, and may transfer a measured cardiac sound signal to the cardiac sound measurement unit 113.

Furthermore, a plurality of the electrocardiogram electrodes 141 may be provided so that they may measure an electrocardiogram signal based on a potential difference. The electrocardiogram pad 140 may be equipped with an adhesive layer so that it can be easily attached to or detached from the body of a testee.

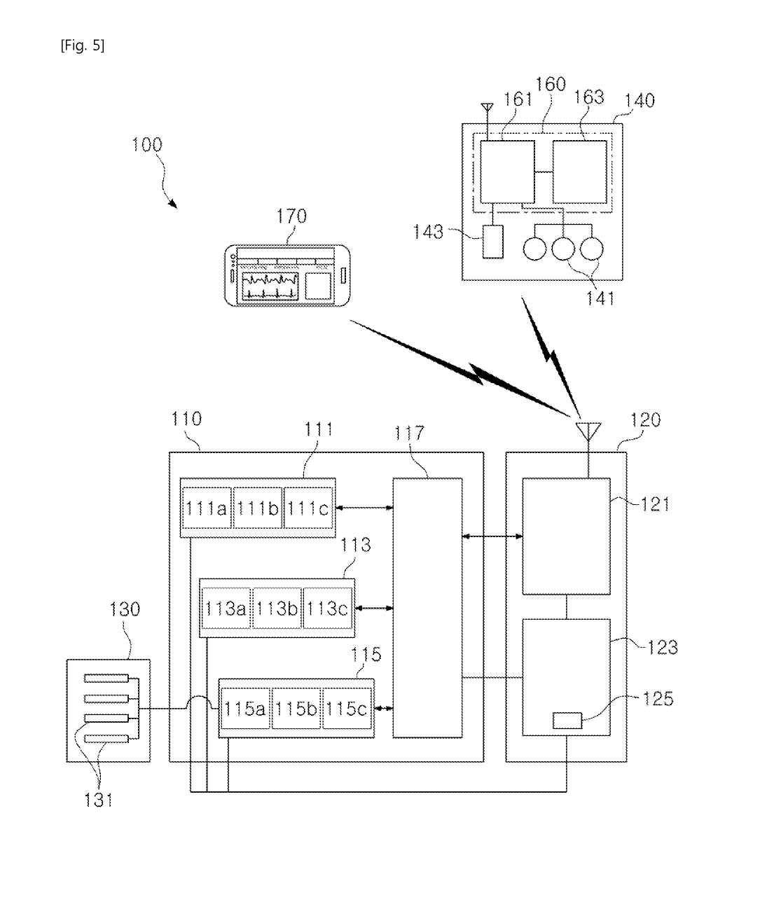

Furthermore, as illustrated in FIG. 5, the electrocardiogram pad 140 may include a second communication power module 160 capable of sending a cardiac sound signal and an electrocardiogram signal to the integrated measurement module 110 and capable of being supplied with power. The second communication power module 160 may include a second wireless communication unit 161 and a second power supply unit 163.

The second wireless communication unit 161 may send electrocardiogram signals, extracted by the electrocardiogram electrodes 141 and the cardiac sound sensor 143, to the electrocardiogram measurement unit 111 and cardiac sound measurement unit 113 of the integrated measurement module 110, respectively.

In this case, the second wireless communication unit 161 may send the cardiac sound signal and the electrocardiogram signal to the cardiac sound measurement unit 113 and the electrocardiogram measurement unit 111 through the first wireless communication unit 121. The second wireless communication unit 161 and the first wireless communication unit 121 may communicate with each other through Wi-Fi, Bluetooth, Zigbee, NFC, WirelessHART, a Body Area Network (BAN), a Wireless BAN (WBAN), or a Personal Area Network (PAN), such as an ultra wideband (UWB), but may communicate with each other over the BAN or PAN.

The second power supply unit 163 may supply power for driving the electrocardiogram electrodes 141, the cardiac sound sensor 143, and the second wireless communication unit 161. A battery may be detachably coupled to the second power supply unit 163 in such a way as to be replaced.

In this case, the battery may be a disposable primary cell or a rechargeable secondary cell.

Furthermore, the second power supply unit 163 may include a warning unit (not illustrated). The warning unit may warn the abnormal state of the battery. The warning unit may be implemented using an LED lamp for visually giving warning or a speaker for acoustically giving warning.

Meanwhile, the second communication power module 160 may be detachably coupled to the electrocardiogram pad 140. In this case, the second communication power module 160 is equipped with a connection terminal electrically connected to the electrocardiogram electrodes 141 and the cardiac sound sensor 143 and coupling means detachably coupled to the electrocardiogram electrodes 141 and the cardiac sound sensor 143.

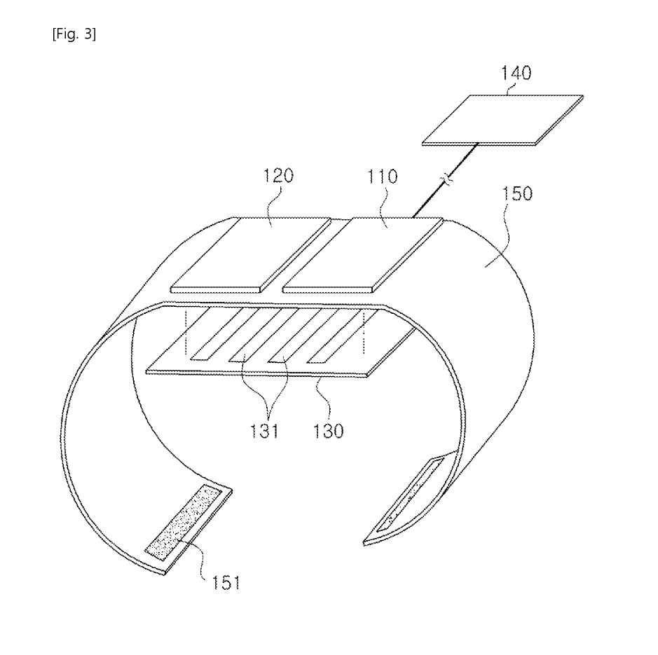

As illustrated in FIG. 3, the apparatus 100 for measuring a pulse wave continuously and automatically in accordance with an embodiment of the present invention may include a cuff 150.

The cuff 150 may receive the integrated measurement module 110 and the communication power module and attach the integrated measurement module 110 and the communication power module to the body of a testee. The cuff 150 may be formed using fabrics.

Meanwhile, the cuff 150 may be configured in the form of a band that surrounds part of the body of a testee, for example, a wrist.

In this case, the cuff 150 may be configured in a circular band having an elastic force or a belt form and may be configured in a form having fastening means 151 both ends of which have fastening structure of male and female forms, for example, a snap button or a Velcro tape.

Furthermore, the integrated measurement module 110 and the first communication power module 120 may be coupled at the central part of the cuff 150.

Meanwhile, the cuff 150 may be integrally formed with the integrated measurement module 110 and the first communication power module 120 may be configured to be detachably attached to the integrated measurement module 110 so that the first communication power module 120 can be replaced in the integrated cuff 150 and integrated measurement module 110.

Furthermore, the bioelectrodes 131 electrically connected to the bioelectrical impedance measurement unit 115 may be provided at the bottom of the cuff 150. The bioelectrodes 131 may be equipped with the biomeasurement pad 130 in such a way as to be detachably coupled at the bottom of the cuff 150.

In this case, if the biomeasurement pad 130 is combined with the cuff 150, it may include a connection terminal for electrically connecting the biomeasurement pad 130 and the integrated measurement module 110 and coupling means for coupling the biomeasurement pad 130 and the cuff 150.

As illustrated in FIGS. 4 to 6, the apparatus 100 for measuring a pulse wave continuously and automatically in accordance with an embodiment of the present invention may include the external terminal 170.

The external terminal 170 may send information about the body of a testee to the integrated measurement module 110 through the first wireless communication unit 121 or may receive information about the state of the cardiovascular system of the testee, checked by the integrated measurement module 110, through the first wireless communication unit 121.

Furthermore, the external terminal 170 may include a testee terminal 171 and a feedback terminal 173.

The testee terminal 171 is a device carried by a testee and may be implemented using a tablet PC or a smart phone. The testee terminal 171 may receive information about the state of the cardiovascular system of a testee checked by the integrated measurement module 110, for example, an electrocardiogram signal, a cardiac sound signal, a bioelectrical impedance signal, and information about a disease of the cardiovascular system, a heartbeat, and blood pressure expected based on the signals and notify the testee of the received information through a display or may receive information about the body of the testee and send the received information to the integrated measurement module 110.

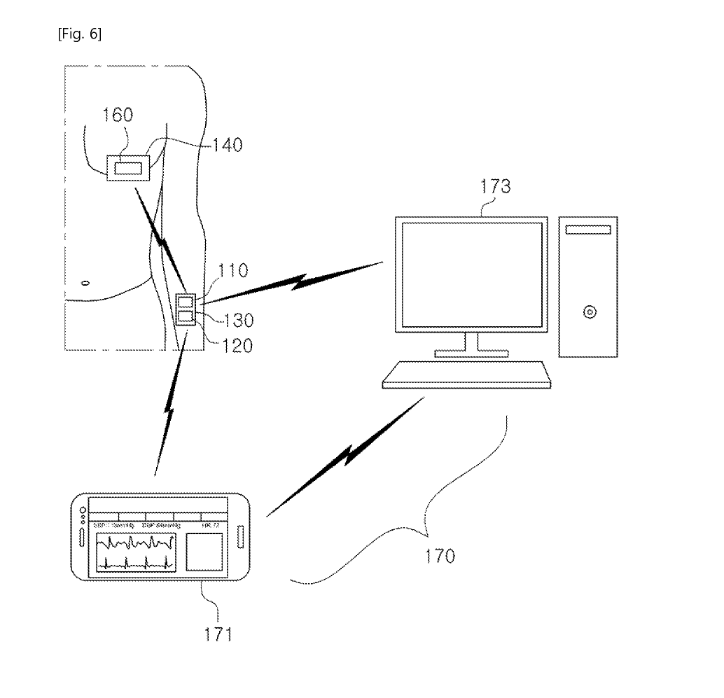

Furthermore, the feedback terminal 173 is a device by which medical personnel may check information about the state of the cardiovascular system of a testee and feed the information back. The feedback terminal 173 may receive information about the state of the cardiovascular system of a testee through the testee terminal 171 or directly receive the information through the first wireless communication unit 121, may notify medical personnel of the received information through a display, may receive feedback information determined by the medical personnel, and may send the received feedback information to the testee terminal 171 (refer to FIG. 6).

Actions and effects between the aforementioned elements are described.

In the apparatus 100 for measuring a pulse wave continuously and automatically in accordance with an embodiment of the present invention, if the integrated measurement module 110 and the first communication power module 120 are fixed to the biomeasurement pad 130, the biomeasurement pad 130 is attached to the wrist portion of a testee so that the bioelectrodes 131 of the biomeasurement pad 130 are brought in contact with the wrist portion of the testee.

In contrast, if the integrated measurement module 110 and the first communication power module 120 are combined with the cuff 150, the cuff 150 is combined with the wrist portion of a testee so that the bioelectrodes 131 are brought in contact with the wrist portion of the testee in the state in which the biomeasurement pad 130 has been combined with the bottom of the cuff 150.

Furthermore, if the electrocardiogram electrodes 141 and cardiac sound sensor 143 of the electrocardiogram pad 140 are attached to a portion in which the heart of a testee is placed and the second communication power module 160 has not been combined with the electrocardiogram pad 140, the electrocardiogram pad 140 and the integrated measurement module 110 are coupled using an electric wire.

In this state, power is supplied to the integrated measurement module 110 through the first power supply unit 123, and an electrocardiogram signal, a cardiac sound signal, and a bioelectrical impedance signal are measured through the electrocardiogram electrodes 141, the cardiac sound sensor 143, and the bioelectrodes 131.

Furthermore, the measured electrocardiogram signal, cardiac sound signal, and bioelectrical impedance signal are provided to the controller 117 through the amplification units 111a, 113a, and 115a, the filtering units 111b, 113b, and 115b, and the conversion units 111c, 113c, and 115c included in the respective measurement units 111, 113, and 115.

In this case, if the electrocardiogram pad 140 is equipped with the second communication power module 160, an electrocardiogram signal measured by the electrocardiogram pad 140 and a cardiac sound signal may be transmitted to the controller 117 through the first wireless communication unit 121 via the second wireless communication unit 161 (refer to FIG. 6).

Meanwhile, the controller 117 checks information about the state of a cardiovascular system, such as the blood pressure of a testee, based on information about the body of the testee and signals received through the first wireless communication unit 121 and sends the checked information about the state of the cardiovascular system to the testee terminal 171 or the feedback terminal 173 through the first wireless communication unit 121.

Furthermore, the feedback terminal 173 receives feedback information from medical personnel based on the received information about the state of the cardiovascular system of the testee and sends the received feedback information to the testee terminal 171. Accordingly, the testee receives the feedback information from the medical personnel and can clearly determine the state of the cardiovascular system.

Accordingly, the apparatus 100 for measuring a pulse wave continuously and automatically in accordance with an embodiment of the present invention can accurately check the state of the cardiovascular system of a testee based on a cardiac sound signal, a bioelectrical impedance signal, and an electrocardiogram signal and can easily check the state of the cardiovascular system regardless of a place because it is reduced in size and can be easily carried.

Furthermore, if blood pressure is to be measured, it can be accurately measured by measuring it based on a cardiac sound signal, a bioelectrical impedance signal, and an electrocardiogram signal.

Furthermore, the state of the cardiovascular system of a testee can be easily measured because the apparatus 100 for measuring a pulse wave continuously and automatically is installed in the state in which it has been worn on the wrist of the testee.

Furthermore, feedback information from medical personnel can be easily received because information about the checked state of a cardiovascular system can be wirelessly transmitted to and received from a testee.

Furthermore, damage attributable to the wrinkling of power lines can be prevented because the electrocardiogram pad 140 and the controller 117 wirelessly exchange pieces of information and power lines can be omitted.

Furthermore, the first wireless communication unit 121 can be repeatedly reused because it is detachably coupled to the integrated measurement module 110.

Furthermore, one stroke volume of the heart can be calculated accurately and easily because the PEP is measured based on a cardiac sound signal and an electrocardiogram signal.

Although the embodiments of the present invention have been described above, the scope of the present invention is not limited to the embodiments and includes all changes and modifications which are easily changed by those skilled in the art to which the present invention pertains from the embodiments of the present invention and recognized to be equivalent.

### INDUSTRIAL APPLICABILITY

The present invention can be used in industry fields related to health, such as a healthcare field and a medical field.

1. An apparatus for measuring a pulse wave continuously and automatically, comprising:
an integrated measurement module comprising an electrocardiogram measurement unit which measures electrocardiogram of a testee, a bioelectrical impedance measurement unit which measures bioelectrical impedance of the testee based on a potential difference, a cardiac sound measurement unit which measures a cardiac sound of the testee, and a controller which measures a state of a cardiovascular system of the testee based on a pulse transit time (PTT′) calculated based on the electrocardiogram signal measured by the electrocardiogram measurement unit, the bioelectrical impedance signal measured by the bioelectrical impedance measurement unit, and the cardiac sound signal measured by the cardiac sound measurement unit;
a first communication power module comprising a first wireless communication unit which is electrically connected to the integrated measurement module and wirelessly sends and receives information of the integrated measurement module and information of an external terminal and a first power supply unit which supplies power to the first wireless communication unit and the measurement module; and
a biomeasurement pad in which the integrated measurement module and the first communication power module are seated and which comprises bioelectrodes electrically connected to the bioelectrical impedance measurement unit.

2. The apparatus of claim 1, wherein the controller calculates the pulse transit time (PTT′) using an equation below:
the pulse transit time (PTT′)=PTT−PEP
(wherein PTT is a time interval between a peak point R in the electrocardiogram signal and a highest point or lowest point in the bioelectrical impedance signal, and the PEP is a time interval between the peak point R in the electrocardiogram signal and a first highest point S1 of the cardiac sound signal).

3. The apparatus of claim 1, further comprising a cuff which fixes the integrated measurement module and the first communication power module in such a way as to surround part of a body of the testee.

4. The apparatus of claim 1, wherein the biomeasurement pad is attached to a wrist portion of the testee.

5. The apparatus of claim 1, wherein the first communication power module is detachably coupled to the integrated measurement module and the cuff and is capable of being reused.

6. The apparatus of claim 1, wherein the first power supply unit comprises a warning unit which warns a state of power supply.

7. The apparatus of claim 1, further comprising an electrocardiogram pad comprising an electrocardiogram electrode which is electrically connected to the electrocardiogram measurement unit and detects the electrocardiogram signal for the testee based on the potential difference and a cardiac sound sensor which is electrically connected to the cardiac sound measurement unit and detects the cardiac sound signal for the testee, wherein the electrocardiogram pad is attached to a portion in which a heart of the testee is placed.

8. The apparatus of claim 7, wherein the electrocardiogram pad comprises a second communication power module comprising a second wireless communication unit which wirelessly sends the electrocardiogram signal and the cardiac sound signal and a second power supply unit which supplies power to the second wireless communication unit, the electrocardiogram electrodes, and the cardiac sound sensor.

9. The apparatus of claim 7, wherein the second communication power module is detachably coupled to the electrocardiogram pad and is capable of being reused.

10. The apparatus of claim 7, wherein the second wireless communication unit communicates with the first wireless communication unit over a Personal Area Network (PAN) or a Body Area Network (BAN).

11. The apparatus of claim 1, wherein the controller receives information about a body of the testee from the external terminal and measures the state of the cardiovascular system of the testee based on the information about the body of the testee.

12. The apparatus of claim 1, wherein the external terminal comprises:
a user terminal used by the testee, and
a tester terminal which directly receives information about the state of the cardiovascular system of the testee through the first wireless communication unit or receives the information through the user terminal, receives feedback information from a tester, and sends the information to the user terminal.

13. A method for measuring blood pressure, comprising steps of:
measuring an electrocardiogram signal of a testee;
measuring a bioelectrical impedance signal of the testee based on a potential difference;
measuring a cardiac sound signal of the testee; and
measuring a state of a cardiovascular system of the testee based on a pulse transit time calculated based on the electrocardiogram signal, the bioelectrical impedance signal, and the cardiac sound signal,
wherein the step of measuring the state of the cardiovascular system of the testee comprises calculating a pulse transit time (PTT′) using an equation below:
the pulse transit time (PTT′)=PTT−PEP
(wherein PTT is a time interval between a peak point R in the electrocardiogram signal and a highest point or lowest point in the bioelectrical impedance signal, and the PEP is a time interval between the peak point R in the electrocardiogram signal and a first highest point S1 of the cardiac sound signal).

14. The method of claim 13, wherein:
the bioelectrical impedance is measured in a wrist portion of the testee, and
the electrocardiogram signal is measured in a heart portion of the testee.

15. An apparatus for measuring a pulse wave continuously and automatically, comprising:
an electrocardiogram measurement unit which measures electrocardiogram of a testee,
a bioelectrical impedance measurement unit which measures bioelectrical impedance of the testee based on a potential difference,
a cardiac sound measurement unit which measures a cardiac sound of the testee, and
a controller which measures a state of a cardiovascular system of the testee based on a pulse transit time (PTT′) calculated based on the electrocardiogram signal measured by the electrocardiogram measurement unit, the bioelectrical impedance signal measured by the bioelectrical impedance measurement unit, and the cardiac sound signal measured by the cardiac sound measurement unit.

16. The apparatus of claim 15, further comprising a biomeasurement pad which comprises bioelectrodes electrically connected to the bioelectrical impedance measurement unit and in which the controller is seated.

17. The apparatus of claim 16, further comprising a power module which is seated in the biomeasurement pad and which supplies power to the controller.

18. The apparatus of claim 15, further comprising a communication unit which is electrically connected to the controller and communicates information about the state of the cardiovascular system of the testee measured by the controller and information inputted through an external terminal with the controller in a wired or wireless manner.
